(19) United States
(12) Patent Application Publication
(10) Pub. No.: US 2026/0262951 A1
(43) Pub. Date: Sep. 10, 2026

(54) DEVICE AND METHOD OF MEASURING HEALTH DATA USING A PORTABLE DEVICE FOR SELF-DIAGNOSIS

(71) Applicant: Ashissh RAICHURA, Surrey (CA)

(72) Inventor: Ashissh RAICHURA, Surrey (CA)

(21) Appl. No.: 17/813,733

(22) Filed: Jul. 20, 2022

Related U.S. Application Data
(60) Provisional application No. 63/223,836, filed on Jul. 20, 2021.

(51) Int. Cl. A61B 5/0205; A61B 5/00; A61B 5/0235; A61B 5/1455; A61B 5/318; G16H 50/20

(52) U.S. Cl. CPC A61B 5/02055; A61B 5/0006; A61B 5/14551; A61B 5/318; G16H 50/20; A61B 5/0235; A61B 2560/0223; A61B 2562/0247; A61B 2562/0295

(57) ABSTRACT

A device and method for point-of-care collection, analysis and diagnosis of users. The device includes sensors to detect health data of the users and uses processors to optimize the recorded health data and provide point-of-care diagnoses. The device may then transmit the health data to hospitals and physicians for further care.

### FIELD OF INVENTION

This invention generally relates to a device and method of measuring health data with a portable device having multiple integrated sensors and a method of providing diagnoses using a point-of-care (POC) device.

### BACKGROUND

Although healthcare is a key element of any modern society, according to the World Health Organization (WHO), over half of the global population lacks access to adequate healthcare. In many countries, healthcare remains privatized, and people are required to pay exorbitant medical bills to receive necessary healthcare. Even in countries with public healthcare, there are limitations on the number of physicians and medical supplies readily available for patients. A key factor contributing to lack of access to adequate healthcare is the absence of readily available early diagnostic and POC devices that can measure and keep track of personal health data. A significant portion of medical costs may be reduced if diseases and conditions are diagnosed at an earlier stage, yet the medical field lacks access to diagnostic and POC devices.

With an increased shortage of physicians and a growing aging population around the globe, there is a serious need for advances in technology for self-diagnostic and POC devices. There is a need to provide efficient, adequate, and cost-effective healthcare for individuals while allowing physicians to keep track of more patients' health data.

Portable diagnostic and POC devices are referred to as technologically advanced devices, which enable a user to perform a diagnosis at the clinic, home, or any remote area. They are wireless communication interfaces with long battery life and various sensors, which are able to collect biological data and process the collected data in order to provide further information to users regarding their health. Such devices facilitate communication between a user and a physician, measure health data such as blood glucose levels, weight, and blood pressure, and record and share the user's health data with a physician or a medical institute in order to maintain the user's medical records, which are further stored in a secured place.

Especially with the current global pandemic due to COVID-19, there is a serious need for POC devices and readily available at-home monitoring devices. Such devices may be used to monitor patients suffering from chronic diseases such as diabetes, cardiac disease and hypertension. It may also be a safer option for patients who are not able to make regular trips to a clinic or a hospital.

Despite the current advances in diagnostic and POC devices, the average consumer does not have access to such POC devices that are readily available, cost-effective, and measure sufficient health data to provide diagnostic information. Patients are required to visit hospitals and clinics to measure health data and multiple visits are usually required to record adequate data to provide proper diagnosis. Additionally, the devices used to record patient data are outdated and are limited to one type of measurement such as, heart rate, blood-glucose levels, or ECG.

As a result, there is a need for a personal portable diagnostic device, which can be used by average consumers to record data, relay data to physicians and health professionals, and provide self-diagnostic functions to provide more efficient analysis of data.

### SUMMARY OF THE INVENTION

The present invention is directed to a device and method for POC health data collection, analysis and diagnosis of users which allow users to readily record health data and provides diagnostics based on physiological basal levels and the user's own basal levels, as set forth in or otherwise apparent from the description and drawings that follow, and that which is learned by the practice of the subject matter disclosed herein.

In one broad aspect of the invention, a POC device for health data collection, analysis and diagnosis of users comprises a housing, a plurality of sensors, an electrode for measuring the user's electrical activity of the heart, a power source, and a processor.

A method of providing POC health data collection, analysis and diagnosis of users comprises the steps of: providing a device for POC testing and diagnostics comprising a housing, a plurality of sensors, an electrode for detecting or measuring the user's electrical activity of the heart, a power source, and a processor, inputting health data by the user, recording user's health data using the device, establishing a physiological basal level of the user, comparing newly recorded data to the user's physiological basal level, and processing deviations of newly recorded data to the physiological basal level to provide the user with diagnoses.

The discussion above may cover only some of the aspects of the invention. Other and sometimes more particular aspects of the invention will be appreciated by reference to the following description of at least one preferred mode for carrying out the invention in terms of one or more examples. The following mode(s) for carrying out the invention are not a definition of the invention itself but are only example(s) that embody the inventive features of the invention.

### BRIEF DESCRIPTION OF THE DRAWINGS

At least one mode for carrying out the invention in terms of one or more examples will be described by reference to the drawings in which:

FIG. 1 shows a front perspective view of an embodiment of a device for measuring electrical activity of the heart with a plurality of sensors.

FIG. 2 shows a bottom perspective view of an embodiment of the device shown in FIG. 1 with an air pump and an electrode.

FIG. 3 shows an embodiment of the device shown in FIG. 1 in use taking electrocardiography measurements.

FIG. 4 shows a top perspective view of an embodiment of the device shown in FIG. 1.

FIG. 5 shows a flowchart of embodiments of the device and its processes for measuring a user's health data using the device of FIG. 1.

FIG. 6 shows an embodiment of the device shown in FIG. 1 in use measuring a person's body temperature.

FIG. 7 shows an illustration showing how the device shown in FIG. 1 is used to measure blood oxygen levels.

FIG. 8 shows a side perspective view of an embodiment of the device shown in FIG. 1 showing a blood strip insertion hole and a charging port on the side surface.

FIG. 9 shows an embodiment of a method of using the device shown in FIG. 1 to measure blood glucose.

FIG. 10 shows an embodiment of the device shown in FIG. 1 in use to measure blood pressure.

FIG. 11 shows an illustration of an embodiment of the device shown in FIG. 1 in use with a user to measure blood pressure.

### DETAILED DESCRIPTION

The present invention is a POC device for health data collection, analysis and diagnosis of users using a plurality of sensors, recording users' health data including but not limited to body temperature, blood pressure, blood oxygen levels, processing such health data to provide preliminary diagnoses, facilitating communication between users and health professionals, and storing a secure record of users' health data for the measurement, processing, communication, storing of health data, and further diagnoses of the users, as discussed above.

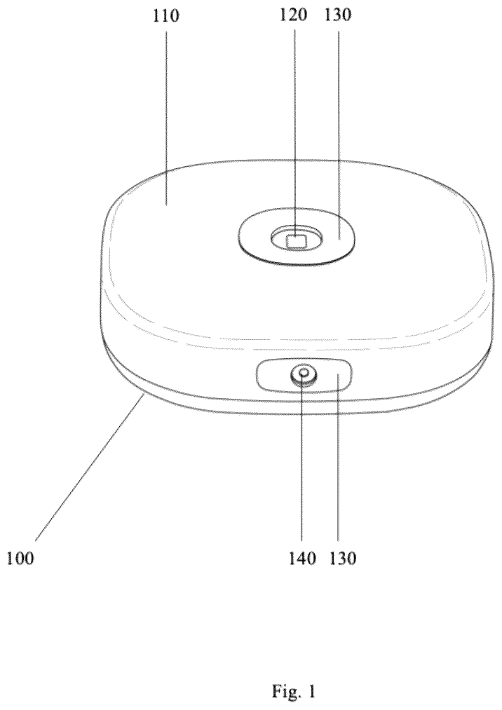

With reference to FIG. 1, a front perspective view of an embodiment of the invention is shown. In one embodiment of the invention, the device 100 has an optical sensor 120, an infrared sensor 140, an electrode (not shown) and a processor (not shown). The sensors 120, 140 may be surrounded by a frame 130, which is optimized such that when a user places a finger or other body part on the sensors 120, 140 there is minimal interference from the external environment. The frame 130 may be composed of steel or any other rigid material that does not interfere with the sensors.

In other embodiments, the device 100 has a plurality of sensors, each specialized to detect and measure specific health data, including but not limited to biosensors, electrochemical sensors, infrared sensors, optical biosensors, immunosensors, electrodes, and accelerometers.

In an embodiment, the device 100 is encapsulated in a housing 110. The housing 110 may have rounded edges. The top and bottom surfaces of the device may be slightly convex or may be convex with flat centres. The shape of the top and bottom surfaces of the housing 110 may also be a squircle, a square with rounded corners. The housing 110 may be made of any light and durable material that does not interfere with the sensors including but not limited to plastic. The shape and size of the housing 110 allows users to hold the device with ease and houses the sensors in such way that optimizes measurements of health data.

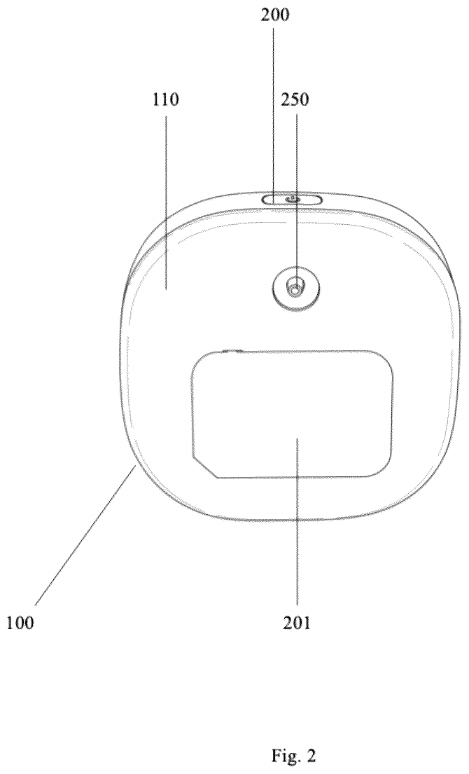

With reference to FIG. 2, in an embodiment, the device 100 has an air pump 250 and an electrode 201. The air pump 250 and the electrode 201 may be on the bottom surface of the device 100. The electrode 201 may be composed of a highly conductive material including but not limited to steel, stainless steel, silver, silver chloride, or graphite.

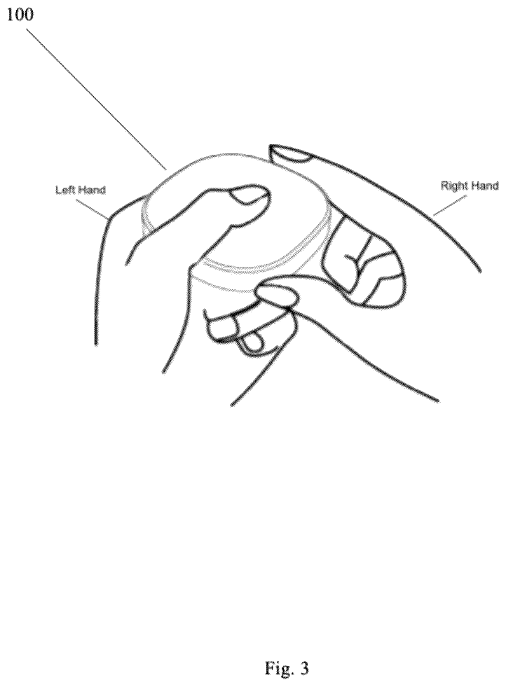

With reference to FIG. 3, in an embodiment, the device 100 is used as an electrocardiogram (ECG) to record electrical activity of a user's heart. In this embodiment, the device 100 uses the electrode 201 to measure the electrical activity of the user's heart. Physiologically, electrical impulses coordinate contractions of different chambers of the heart to pump blood throughout the body. The strength and timing of such electrical impulses may be measured at different points of the body to show the how fast and strong the heart is beating and the rhythm of such beats. The electrode 201 acts as a sensor for the electrical impulses and records the changes in current over time. The device 100 may be used to measure the electrical activity of the heart at multiple points of the body to optimize the ECG recording. Recordings from different parts of the body pick up electrical activity from a different position of the heart muscle, which allows a more accurate reading once such data is analyzed and interpreted.

In an embodiment, to further optimize the ECG recordings, the device 100 uses the electrode 201 to measure the electrical signal of the user's heart at various points of the body and optimizes the electrical signal recording using the infrared sensor 140 and the optical sensor 120. To measure the electrical activity of the user's heart, the user may hold the device 100 in the user's left hand with his or her thumb on the optical sensor 120. The user may then place the index and middle fingers of the user's left hand on the electrode on the bottom surface of the device. The device 100 may be held in such way that the infrared sensor is pointing away from the user. Then, the user may place the index finger of the user's right hand on the infrared sensor. The user may place both hands on his or her lap but may not have the two hands touching each other. The device 100 may then record the user's electrical activity of the heart. In such embodiment, the device 100 concurrently measures the user's body temperature using the infrared sensor 140 and the SpO2 using the optical sensor 120, while measuring the electrical activity of the user's heart. A person's heart activity causes minor rhythmic changes to a person's pulse and blood flow, and this pulse and rhythm changes may be detected with the infrared sensor 140 and the optical sensor 120 of the device 100. The infrared sensor 140 detects minor changes in body temperature and the optical sensor 120 detects the minor changes in the oxygen levels in the blood. The pulse and rhythm detected by the infrared sensor 140 and the change in body temperature by the optical sensor 120 is then compared with the electrical activity recorded by the electrode 201. If the pulse and rhythm of the three recordings are aligned, then the ECG recording is likely highly accurate. If the pulse and rhythm are not aligned, the ECG recording is likely inaccurate and requires further processing or optimization. In an embodiment, the device 100 may further comprise a processor with a machine learning and optimization algorithm and artificial intelligence to optimize the ECG recordings by comparing the ECG recordings to the infrared sensor 140 and optical sensor 120 records.

In an embodiment of the invention, the ECG recordings may be further optimized by the device 100 further comprising a processor with a machine learning and artificial intelligence algorithm that detects abnormalities in a person's heart activities. Such abnormalities may be, but is not limited to, predicted heart rate, predicted heart rate variability (HRV), heart rhythm, heart rate type, number of missing heartbeats, location of missing heartbeats, AFIB (Atrial fibrillation), sinus arrhythmia, irregular heart internal (time), QRS wave (interval), PR wave (Interval), QRSD, QTC, sleep apnea, ventricular fibrillation, ventricular flutter, and atrial flutter. The device 100 analyses the ECG recordings with the machine learning algorithm and may diagnose for heart conditions.

Upon recording of ECG data, the device 100 may then display or transmit the ECG recording of the user to a third party, such as the user's clinic or physician. The recorded data may also be further analyzed by a processor, the mobile application or computer software to diagnose the user.

In an embodiment, the user may have other parts of the body come in contact with the electrode for additional ECG recordings. Additional ECG recordings may be performed by holding the device in the above-described manner, removing the left index and middle fingers from the electrode and then placing the electrode on a different body part such as the chest or thigh. The device may then measure the electrical activity of the heart from multiple leads and record a more accurate ECG recording.

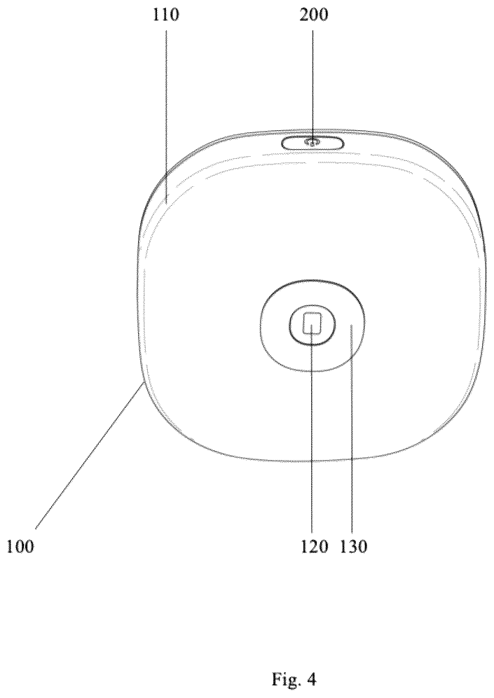

With reference to FIG. 4, in an embodiment of the invention, the device 100 has an optical sensor 120, a housing 110, and a power button 200 which controls the power (i.e., ON/OFF status) to the device 100. In an embodiment, the device 100 has a plurality of buttons 200 with each button having a different function including but not limited to controls for each sensor and wireless connectivity.

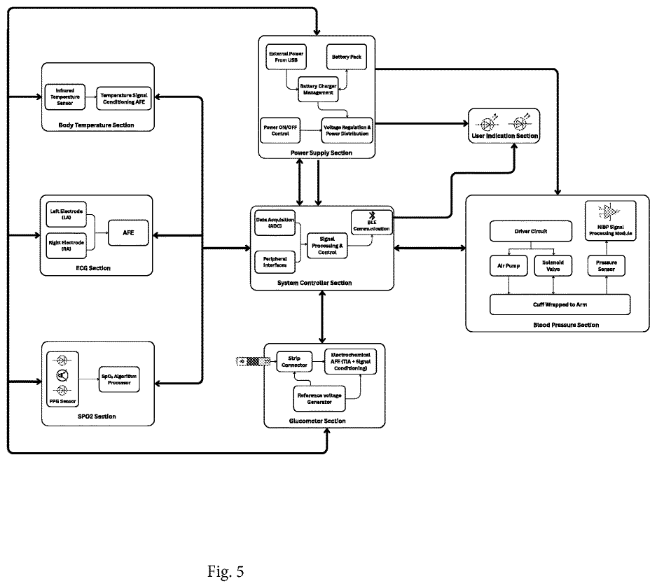

With reference to FIG. 5, an embodiment of the device 100 has a power supply section 501, ECG/EKG section 502, a SpO2 section 503, a blood-based strip vitals section 504, a blood pressure section 505, and a processor 560. The power supply section 501 has a battery 510 and a voltage regulator 511. The battery 510 may be recharged using external power from USB 512 via a battery charging circuit 513. The power supply section 501 supplies power to the device 100 and its individual components.

In an embodiment, the ECG/EKG section 502 may have a plurality of electrodes 520 and an analog front end 521 having analog to digital converters, amplifiers, and filters to process data received by the sensors in a digital format. The analog front end 521 is customizable for each type of sensor on the device 100 for optimal measurements.

In an embodiment, the SpO2 section 503 has light emitting diodes (LEDs) 530, a photo diode 531 and an analog front end 532. The device 100 has a plurality of LEDs 530 capable of emitting light at multiple wavelengths, for example, for SpO2 measurements, a red light at approximately 650 nm wavelength and an infrared light at approximately 950 nm. The photo diode 531 detects light and converts it to an electrical signal, which is further processed by the analog front end 532.

In an embodiment, the blood-based strip vital section 504 has a blood strip 540, a converter 542 and an amplifier 543. In an embodiment, the blood strip 540 contains a capillary or a surface onto which the user's blood may be placed. The blood strip 540 may contain an enzyme, which reacts with the compound in the blood to be detected. The enzyme facilitates a chemical reaction that has an effect on the current that may run through the blood strip 540. The device 100 compares the current running through the blood strip 540 to a reference voltage 544 and the difference in current may be recorded and further processed by the converter 542 and the amplifier 543.

In an embodiment, the device 100 may be used to detect other components in the user's blood including but not limited to hemoglobin, total cholesterol, blood group lipid profile, hba1c, triglyceride, uric acid, ketones, renal function (renal panel) liver function, kidney function, thyroid, vitamin d3, white blood cells, and neutrophils.

In an embodiment, the blood pressure section 505 has an air pump 550, a solenoid valve 551, an arm cuff 552, and a pressure sensor 553. A driver circuit 555 controls the activity of the air pump 550 and the solenoid valve 551. For blood pressure measurements, the device 100 may be removably attached to the arm cuff 552 so that the device 100 is used liked a sphygmomanometer. The air pump 550 inflates the arm cuff 552 to increase pressure of air and the solenoid valve 551 releases air to decrease pressure of air. The pressure sensor 553 detects the pressure and the changes in pressure and the pressure measurement may be amplified by an amplifier 554.

In an embodiment, the device 100 may wirelessly link to a mobile device 570 and an application on the mobile device 570 or a computer 580 and computer software using a processor 560. The processor 560 may comprise a transmitter to transfer data wirelessly. The user may also control the device 100 and its functions using the application and track information related to the device 100 such as its battery and performance levels. The user may also input personal health data including, but not limited to, age, height, gender, and medical history, which may be used to optimize processing of data and measurements recorded by the device 100. Once the device 100 is used to measure the user's health data, the recorded data may be transmitted to the mobile device 570 for processing. The processor 560 may also be used to process recorded data. The recorded data is collected, processed and analyzed by the processor 560 using the user's personal health information and previous measurements to provide the user with more accurate measurements. The processed data may then be stored on the mobile device 570 or an external database to keep track of the user's medical data. The data may be shared with a physician or a health clinic for further record keeping, analysis and diagnoses. The data may also be further analyzed by the processor to provide diagnoses to the user.

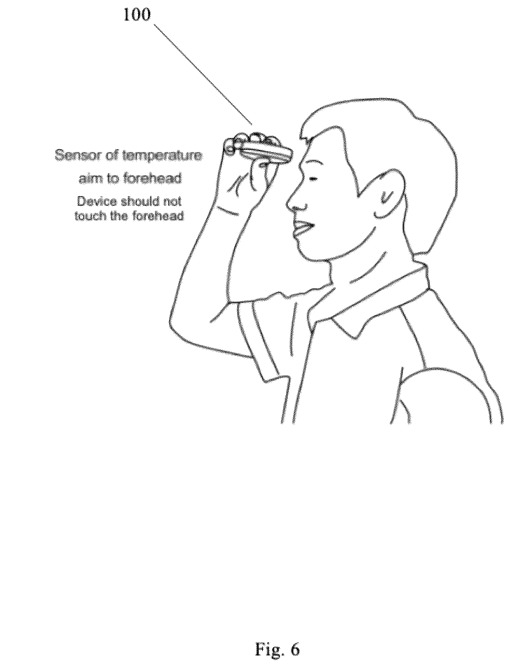

With reference to FIG. 6, the device 100 may be used to measure the user's body temperature. In an embodiment, the device 100 has an infrared sensor (not shown), which is used to measure the user's external temperature. The user may place the device 100 approximately one to two centimeters away from an exposed body part, for example, the user's forehead or armpit. The infrared sensor detects infrared energy from the user's body part and relays the data to a processor or a processor (now shown). In embodiments of the invention, other sensors and components such as a thermistor may be used to detect the user's body temperature. Once the user's temperature is recorded and processed, the device 100 may transmit the information to the mobile device 570 to be stored and displayed. In an embodiment, the user's recorded body temperature data may be used for further analysis and diagnosing the user.

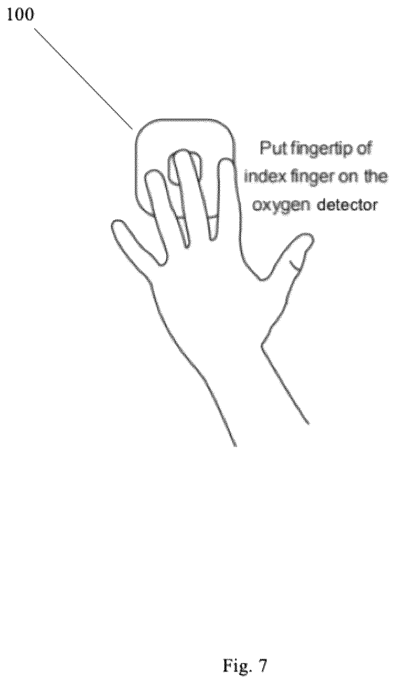

With reference to FIG. 7, in an embodiment, the device 100 may be used to measure the oxygen saturation level in the user's blood (SpO2). In an embodiment, the device 100 has LEDs 230 and an optical sensor 120, which may be used to detect the user's SpO2. To measure SpO2 using the device 100, the user may place the tip of the user's finger on the optical sensor 120. The LEDs 530 emit light at specified wavelengths and the optical sensor 120 detects the intensity of light. Since oxygenated red blood cells (hemoglobin) and non-oxygenated hemoglobin absorb light at different wavelengths, SpO2 may be measured by measuring the intensity of light after absorption. Once the SpO2 data is recorded and processed, the device 100 may transmit the information to the mobile device 570 to be stored and displayed.

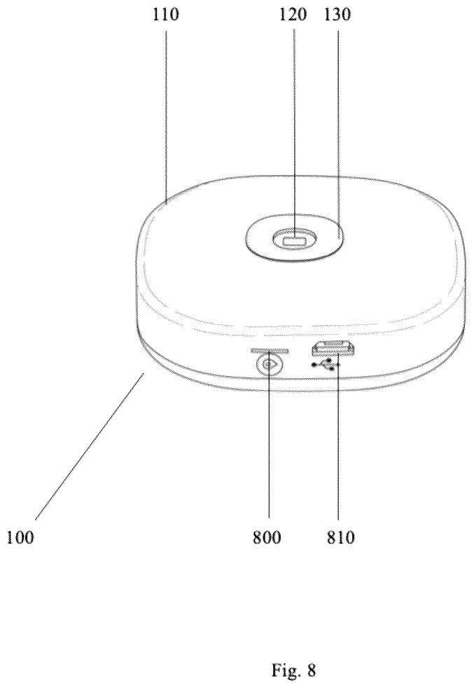

With reference to FIG. 8, in an embodiment of the invention, the device 100 has a USB charging port 810 and a blood strip insertion hole 800. The USB charging port may be used to charge the battery 510.

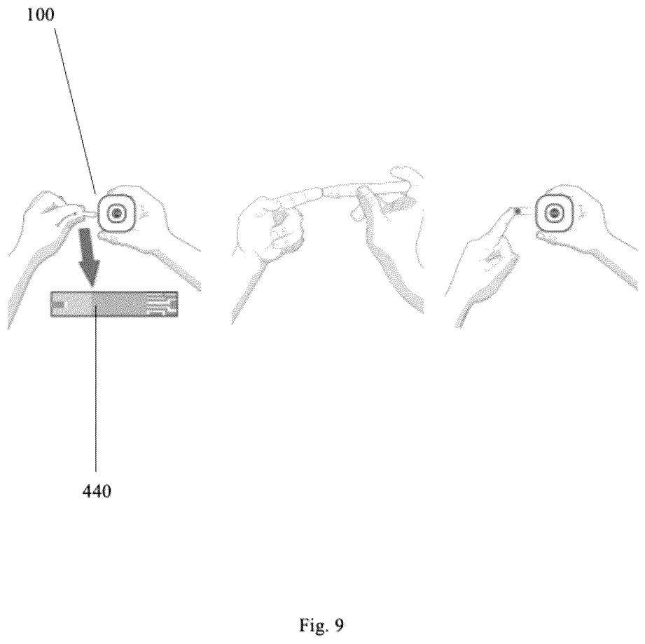

With reference to FIG. 9, in an embodiment, the device 100 may be used for POC blood testing. The user may place his or her blood on the blood strip 540, which may have a capillary or an absorptive surface. The blood strip 540 has a plurality of enzymes, which reacts with specific compounds in the user's blood. The enzymatic reaction may facilitate an electrical, chemical, or visual signal, which may be detected by a sensor on the device 100.

In an embodiment, the device 100 measures a user's blood glucose levels. The user places his or her blood on the blood strip 540. The blood strip 540 contains ferricyanide and an enzyme, including but not limited to glucose oxidase, which is capable of oxidizing glucose to gluconic acid. Gluconic acid reacts with ferricyanide on the blood strip 540 to form ferrocyanide, which allows electrical current to flow through the blood strip 540. The strength of the current is directly related to the concentration of ferrocyanide produced by the glucose in the user's blood. The blood strip 540 may be inserted into the blood strip insertion hole 700 and the device 100 may measure the electrical current that flows through the blood strip 540. Depending on the amount of glucose in the blood, the amount of ferrocyanide on the blood strip 540 will differ, causing a difference in the current. The blood-based strip vital section compares the current to a reference voltage 544. The difference in current may be recorded and further processed by the converter 542 and the amplifier 543. The device 100 may then transmit the recorded blood glucose level of the user to the user's clinic or physician. The recorded data may also be further analyzed by the mobile application or computer software to diagnose the user.

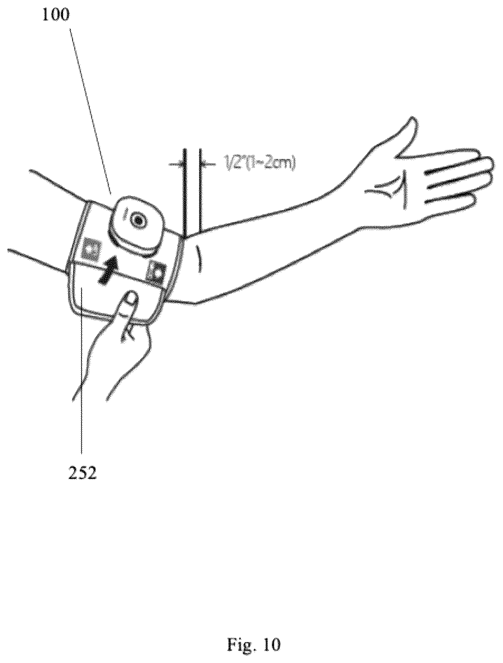

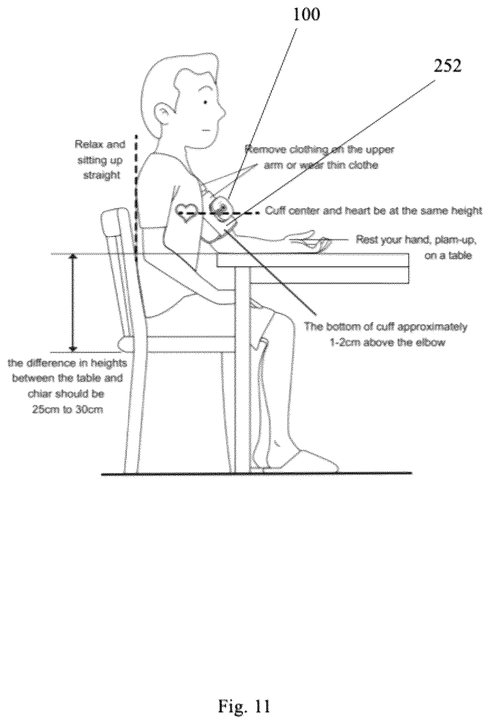

With reference to FIGS. 10 and 11, in an embodiment, the device 100 may be used to measure and record a user's blood pressure. To measure and record blood pressure of the user, the device 100 may be removably attached to an arm cuff 552 by an attachment mechanism so that the device 100 is used like a digital sphygmomanometer. For optimal blood pressure measurements with the device 100, the user may sit upright on a chair by a table, remove clothing on the upper arm or wear thin clothing, have the user's hand on a table with the palm facing upwards, and wear the arm cuff 552 on the user's upper arm at the same height as the user's heart. The arm cuff 552 may be strapped on the user's arm approximately one to two centimeters above the elbow. The air pump 550 inflates the arm cuff 252 so that when the arm cuff 252 is fully inflated, blood cannot flow through the user's brachial artery. Then, the solenoid valve (not shown) releases air from the arm cuff 552 and as pressure in the arm cuff 252 falls below the pressure in the brachial artery, blood will start to flow in the user's arm. The pressure sensor (not shown) detects the pressure and the changes in pressure in the arm cuff 252. The device 100 may then display or transmit the recorded blood pressure of the user to a third party, such as the user's clinic or physician. The recorded data may also be further analyzed by the mobile application or computer software to diagnose the user.

In an embodiment, the device 100 may comprise the application of artificial intelligence and machine learning to interpret and process recorded data of the user. Users may input data such as age, gender, nationality, level of physical activity, which may be used to establish a physiological baseline for the user. Then, the device 100 records health data of the user using a plurality of sensors, as described above. The device 100 may then compare the recorded data to the physiological baseline as well as establishing a custom basal level specific to the user. AI and machine learning systems may be used to analyze each set of recorded data or a combination of recorded data sets to establish the user's basal level, to generate risk profiles for the user predicting the probability and time frames for various diseases, and to provide diagnoses and recommendations to improve the user's health.

In an embodiment, the device 100 comprises a plurality of sensors, each with its own optimal performance level. The device 100 may further comprise a processor with a self-calibration machine learning and artificial intelligence algorithm to optimize the performance of its sensors. The performance level of each component may be measured by such processor, usually when the device is powered on, by comparing its optical performance level to its current performance level. If a component's performance level is below a certain threshold, the device 100 shall notify the user. For example, if performance level of a sensor is below 90%, the device 100 may notify the user as well as the system operator through a mobile application or computer software.

In an embodiment, the device 100 further comprises a processor with a machine learning and artificial intelligence algorithm to optimize the accuracy of the measured health data and to diagnose the user. The recorded health data may be compared to a database of physiological health data or to the user's previously recorded health data. The processor may also compare the recordings of the various sensors of the device 100 to optimize the accuracy of its recordings and to diagnose the user. For example, the recordings of the infrared sensor 140 and optical sensor 120 records may be used to optimize the ECG recordings of the device 100 based on the machine learning and artificial intelligence algorithm.

In an embodiment, the method of diagnosing the user comprises providing the device 100 comprising at least one sensor able to record or measure the user's health data, the processor 560, inputting health data by the user, recording user's health data using the device, establishing a physiological basal level of the user, comparing newly recorded data to the user's physiological basal level, and processing deviations of newly recorded data to the physiological basal level to provide the user with diagnoses.

In an embodiment, the method may further comprise providing the device 100 comprising sensors to record the user's health data including but not limited to heart rate, body temperature, electrical activity of the heart, blood glucose, blood oxygen levels, etc. The user may also input data such as age, gender, nationality, level of physical activity, which the invention may use to establish a physiological baseline for the user. The device 100 may also use recorded data to provide a more accurate physiological baseline of the user.

If new recordings measured by the device 100 for the user significantly deviate from the user's physiological baseline determined by the device 100, the device 100 may further use such deviations to analyze and determine the condition of the user. Prolonged or regular deviation from the physiological baseline may be indicative of a disease or condition. Depending on the type of recorded data and its deviation, the processor 560 may process the data to provide diagnoses for the user. Additionally, based on the length of deviation and the severity of deviation, the device 100 may provide recommendations and suggestions to improve the user's health. The device 100 may also generate risk profile for users and provide the probability and time frames for various diseases.

In an embodiment of the invention, the data collected, and diagnoses generated by the method may be transmitted to a mobile device 570 or a computer 580 for further processing and data collection. The data may also be shared with physicians for further analysis.

1. A point-of-care device for health data collection, analysis and diagnosis of users, comprising:
a housing;
one or more electro-cardiogram (ECG) electrodes to measure the electrical activity of the user's heart;
one or more sensor for detecting or measuring the user's health data; and
one or more processors.

2. The device of claim 1 further comprising a processor with a machine learning algorithm and artificial intelligence to self-calibrate the sensors for detecting or measuring the user's health data.

3. The device of claim 1 further comprising a processor with a machine learning algorithm and artificial intelligence to optimize the accuracy of the measured health data and to diagnose the user.

4. The device of claim 1 further comprising a blood strip insertion hole and blood strips to detect components in the user's blood.

5. The device of claim 1 further comprising a light emitting diode (LED) and an infrared sensor to detect rhythmic changes in body temperature and to optimize the recording of the electrical activity of the user's heart.

6. The device of claim 1 further comprising an LED and an optical sensor to detect rhythmic changes in blood oxygen levels and to optimize the recording of the electrical activity of the user's heart.

7. The device of claim 1 further comprising:
an air pump;
a valve;
a pressure sensor; and
an attachment mechanism for attaching the housing to an arm cuff for measuring the user's blood pressure.

8. The device of claim 1 where the processor in the device further comprises a transmitter for wireless connection with mobile devices and computers.

9. A method of providing point-of-care health data collection, analysis and diagnoses of users, the method comprising the steps of:
providing a device for point-of-care testing and diagnostics, the device comprising:
a housing;
one or more electro-cardiogram (ECG) electrodes to measure the electrical activity of the user's heart;
at least one sensor for detecting or measuring the user's health data;
a power source; and
one or more processors;
inputting health data by the user;
recording user's health data using the device;
optimizing the accuracy of the recorded health data;
establishing a physiological basal level of the user;
comparing newly recorded data to the user's physiological basal level and other average physiological levels; and
processing deviations of newly recorded data to the physiological basal level to provide the user with accurate health data measurements and diagnoses.

10. The method of claim 9 wherein the device further comprises a processor with a machine learning algorithm and artificial intelligence to self-calibrate the sensors for detecting or measuring the user's health data.

11. The method of claim 9 wherein the device further comprises a processor with a machine learning algorithm and artificial intelligence to optimize the accuracy of the measured health data.

12. The method of claim 9 wherein the device further comprises a blood strip insertion hole and blood strips to detect components in the user's blood.

13. The method of claim 9 wherein the device further comprises a light emitting diode (LED) and an infrared sensor to detect rhythmic changes in body temperature and to optimize the recording of the electrical activity of the user's heart.

14. The method of claim 9 wherein the device further comprises an LED and an optical sensor to detect rhythmic changes in blood oxygen levels and to optimize the recording of the electrical activity of the user's heart.

15. The method of claim 9 wherein the device further comprises:
an air pump;
a valve;
a pressure sensor; and
an attachment mechanism for attaching the housing to an arm cuff to measure the user's blood pressure.

16. The method of claim 9 wherein the one or more processors further comprise a transmitter for wireless connection with mobile devices and computers.
